(19) United States
(12) Patent Application Publication
(10) Pub. No.: US 2007/0190528 A1
(43) Pub. Date: Aug. 16, 2007

(54) Method of judging imflammatory disease by using single nucleotide polymorphism in galectin -2 gene

(75) Inventors: Toshihiro Tanaka, Tokyo (JP); Yozo Ohnishi, Tokyo (JP); Kouichi Ozaki, Tokyo (JP); Aritoshi Iida, Kanagawa (JP); Masatsugu Hori, Hyogo (JP); Yusuke Nakamura, Kanagawa (JP)

Correspondence Address: GREENBLUM & BERNSTEIN, P.L.C., 1950 ROLAND CLARKE PLACE, RESTON, VA, 20191, US

(73) Assignees: Toshihiro Tanaka, Tokyo (JP); Yozo Ohnishi, Tokyo (JP); Kouichi Ozaki, Tokyo (JP); Aritoshi Iida, Kanagawa (JP); Masatsugu Hori, Osaka (JP); RIKEN, Saitama (JP)

(21) Appl. No.: 10/568,695

(22) PCT Filed: Aug. 18, 2004
(86) PCT No.: PCT/JP04/12151

§ 371 Date: Nov. 21, 2006

(30) Foreign Application Priority Data
Aug. 18, 2003 (JP) 2003-207698

(51) Int. Cl. C12Q 1/68; C07H 21/04

(52) U.S. Cl. 435/6; 536/23.5

(57) ABSTRACT

An object of the present invention is to identify a novel single nucleotide polymorphism (SNP) associated with the onset and development of inflammatory diseases such as myocardial infarction. The present invention provides a method for judging inflammatory diseases which comprises detecting at least one gene polymorphism in the galectin-2 gene.

### TECHNICAL FIELD

The present invention relates to a method for diagnosing inflammatory diseases comprising detecting the gene polymorphisms in the galectin-2 gene, oligonucleotides used for such method, a diagnostic kit for inflammatory diseases comprising such oligonucleotides, and applications thereof.

### BACKGROUND ART

In spite of changes in lifestyle and novel pharmacological approaches, coronary artery diseases including myocardial infarction are the leading causes of death in the world (Breslow, J. L., Nature Med. 3, 600-601, 1997; Braunwald, E., N. Engl. J. Med., 337, 1360-1369, 1997). Accordingly, identification of genetic and environmental factors associated with the onset of such diseases has been strongly desired.

A common genetic variation is known to be deeply associated with the risk of affliction with lifestyle-related diseases such as diabetes mellitus or hypertension (Risch, N., et al., Science, 273, 1516-1517, 1996; Collins, F. S., et al., Science, 278, 1580-1581, 1997; Lander, E. S., et al., Science, 274, 536-539, 1996). Susceptibility genes for polygenic diseases are identified by a method that involves the use of “genetic linkage” and by a method that involves the use of “association.” Via analysis of genetic linkage, whether or not the locus of the disease susceptibility gene is linked to the locus of the gene marker (mainly microsatellites) is detected, i.e., the relationship between the loci is inspected. In analysis of association, the type (allele) of gene marker (mainly single nucleotide polymorphisms, i.e., SNPs) associated with a disease is detected, i.e., the relationship between alleles is inspected. Accordingly, it can be said that association analysis, which involves the use of a common variation as a marker, is more reliable than genetic linkage analysis which involves the inspection of the localization of disease-associated genes. Single nucleotide polymorphisms (SNPs) are useful polymorphism markers when searching for genes associated with the incidence of a disease or drug reactivity. SNPs may directly influence the quality or quantity of gene products or may increase the risk of serious side effects resulting from given diseases or drugs. Thus, search of a larger number of SNPs may contribute to the identification of disease-associated genes or the establishment of a diagnostic method that prevents side effects that result from the use of drugs.

The correlation between genetic variation and myocardial infarction has been heretofore evaluated by, for example, a method whereby the polymorphisms of the human prostacyclin synthase gene are analyzed to determine genetic factors of myocardial infarction (JP Patent Publication (Kokai) No. 2002-136291 A). However, the genetic variation associated with myocardial infarction has not yet been fully elucidated.

At present, 10 types of mammalian galectins are known. Among those, galectin-2 exhibits high homology of 43% to gelactin-1. As in the case of galectin-1, galectin-2 forms a noncovalent dimer consisting of 2 subunits of 14 kDa, and it undergoes spontaneous agglutination and loses its activity in the absence of a reducing agent. Compared with galectin-1, distribution of galectin-2 in tissues is narrower. Abundant galectin-1 is present in a variety of cell lines such as mesenchymes including muscles; however, a large amount of galectin-2 is primarily observed in the epitheliums of the lower small intestine in normal adult tissues. Functions of galectin-2 have not yet been elucidated (Trends in Glycoscience and Glycotechnology, Vol. 9, No. 45, 1997, pp. 87-93).

### DISCLOSURE OF THE INVENTION

An object of the present invention is to identify a novel single nucleotide polymorphism (SNP) associated with the onset and development of inflammatory diseases such as myocardial infarction. Another object of the present invention is to provide a method for diagnosing inflammatory diseases such as myocardial infarction or a method for developing a therapeutic agent for inflammatory diseases utilizing an identified SNP.

The present inventors have conducted concentrated studies in order to attain the above objects. As a result, they discovered that the gene products of galectin-1 and galectin-2 bind to the myocardial-infarction-susceptible gene product, i.e., lymphotoxin-α (LTA), and that a novel single nucleotide polymorphism (SNP) in the galectin-2 gene is associated with the onset and development of myocardial infarction. This has led to the completion of the present invention.

Thus, the present invention provides a method for judging inflammatory diseases which comprises detecting at least one gene polymorphism in the galectin-2 gene.

Preferably, the present invention provides a method for judging inflammatory diseases which comprises detecting at least one single nucleotide polymorphism in the galectin-2 gene.

More preferably, the present invention provides a method for judging inflammatory diseases which comprises detecting the C/T polymorphism at nucleotide 3279 in the nucleotide sequence of intron 1 of the galectin-2 gene as shown in SEQ ID NO: 1.

Preferably, the inflammatory disease is myocardial infarction.

Another aspect of the present invention provides an oligonucleotide that can hybridize to a sequerice consisting of at least 10 continuous nucleotides including the nucleotide 3279 in the nucleotide sequence of intron 1 of the galectin-2 gene as shown in SEQ ID NO: 1 or a complementary sequence thereof, and that can be used as a probe in the method of any of claims 1 to 4.

A further aspect of the present invention provides an oligonucleotide that can amplify a sequence consisting of at least 10 continuous nucleotides including the nucleotide 3279 in the nucleotide sequence of intron 1 of the galectin-2 gene as shown in SEQ ID NO: 1 and/or a complementary sequence thereof and that can be used as a primer in the method of any of claims 1 to 4.

Preferably, the primer is a forward and/or reverse primer.

A further aspect of the present invention provides a diagnostic kit for inflammatory diseases which comprises at least 1 oligonucleotide according to any of the aforementioned oligonucleotides. Preferably, the inflammatory disease is myocardial infarction.

A further aspect of the present invention provides a method for analyzing the state of galectin-2 expression which comprises detecting the C/T polymorphism at nucleotide 3279 in the nucleotide sequence of intron 1 of the galectin-2 gene as shown in SEQ ID NO: 1.

A further aspect of the present invention provides a method for screening for a therapeutic agent for inflammatory diseases which comprises steps of analyzing the expression level of the galectin-2 or galectin-1 genes in cells in the presence of a candidate substance and selecting a substance that alters such expression level. Preferably, the present invention provides a method for screening for a therapeutic agent for inflammatory diseases which comprises steps of analyzing the expression level of the galectin-2 or galectin-1 genes in cells in the presence of a candidate substance and selecting a substance that increases such expression level.

A further aspect of the present invention provides a method for screening for a therapeutic agent for inflammatory diseases which comprises steps of assaying the binding between lymphotoxin-α (LTA) and the gene product of galectin-2 or galectin-1 in the presence of a candidate substance and selecting a substance that inhibits such binding.

A further aspect of the present invention provides a method for assaying transcriptional activity of galectin-2 which comprises introducing a galectin-2 gene fragment containing the C/T polymorphism at nucleotide 3279 in the nucleotide sequence of intron 1 of the galectin-2 gene as shown in SEQ ID NO: 1 into cells, culturing the cells, and analyzing the expression of such gene.

A further aspect of the present invention provides a method for screening for a substance that inhibits or promotes transcriptional activity of galectin-2 which comprises introducing a galectin-2 gene fragment containing the C/T polymorphism at nucleotide 3279 in the nucleotide sequence of intron 1 of the galectin-2 gene as shown in SEQ ID NO: 1 into cells, culturing the cells in the presence of a candidate substance that inhibits or promotes transcriptional activity of galectin-2, and analyzing the expression of such gene.

A further aspect of the present invention provides a substance that inhibits or promotes transcriptional activity of galectin-2 which is obtained by the aforementioned screening method.

According to the method of the present invention, preferably, a transcription unit comprising a reporter gene ligated to a site downstream of the aforementioned galectin-2 gene fragment is introduced into cells, such cells are cultured, and reporter activity is assayed to analyze the expression of such gene. More preferably, the reporter gene is the luciferase gene.

A further aspect of the present invention provides a method for screening for a transcription-regulatory factor of galectin-2 which comprises bringing a galectin-2 gene fragment containing the C/T polymorphism at nucleotide 3279 in the nucleotide sequence of intron 1 of the galectin-2 gene as shown in SEQ ID NO: 1 into contact with a sample which is assumed to comprise the transcription-regulatory factor of galectin-2 and detecting the binding between the aforementioned gene fragment and the transcription-regulatory factor. Preferably, detection is carried out by gel-shift assay.

### BRIEF DESCRIPTION OF THE DRAWINGS

FIG. 1 shows the results of an experiment to inspect the binding between LTA and galectin-1 or galectin-2 in vitro.

FIG. 2 shows the results of inspecting the influence of SNP (3279C>T) in intron 1 of the galectin-2 gene on transcriptional activity.

FIG. 3 shows the results of inspecting the interaction between galectin-2 and a microtubule, wherein: “a” shows the isolation of TAP-tagged galectin-2 from interacting proteins; “b” shows the results of a coimmunoprecipitation experiment involving endogenous α-tubulin and FLAG-tagged galectin-2 or LTA; and “c” shows the results of an experiment to inspect the colocalization of endogenous α-tubulin and endogenous galectin-2 or LTA in U937 cells.

FIG. 4 shows the results of inspecting expression and colocalization of galectin-2 and LTA in coronary atherectomy specimens, wherein: “a” shows a specimen stained with anti-human LTA; “b” shows a specimen stained -with anti-human galectin-2; “c” shows a specimen stained with monoclonal anti-SMC α-actin; “d” shows a specimen stained with monoclonal anti-CD68; “e” shows a specimen dually-stained with anti-LTA antibody and anti-galectin-2 antibody; and “f” shows a specimen stained with anti-human LTA.

### PREFERRED EMBODIMENTS OF THE INVENTION

In the present invention, the gene products of galectin-1 and galectin-2 were found to bind to the lymphotoxin-α (LTA) product known as a gene product susceptible to inflammatory diseases such as myocardial infarction (Ozaki, K. et al., Nature Genetics 32, 650-654, 2002). Further, a novel single nucleotide polymorphism (SNP) in the galectin-2 gene was identified, the group of myocardial infarction patients and the control group each consisting of approximately 2,000 individuals were subjected to typing via the PCR/DNA sequencing method, and statistical analysis (e.g., the chi-square test) was carried out. As a result, the frequency of the novel SNP was found to be statistically significantly low in myocardial infarction patients. Further, it was found by an experiment involving luciferase assay that this novel SNP has biological functions, and change in the amount of the gene product of galectin-2 causes various inflammatory diseases including, but not limited to, myocardial infarction.

As described above, the galectin-1 and galectin-2 proteins were identified as novel molecules that bind to LTA protein in the present invention. Further, a novel SNP in the galectin-2 gene was identified to have biological functions and to be associated with diseases such as myocardial infarction. Accordingly, use of the novel SNP in the galectin-2 gene that was identified by the present invention enables the development of novel diagnostic and preventive methods for inflammatory diseases such as myocardial infarction and therapeutic agents therefor. Hereafter, embodiments of the present invention are described in greater detail.

[1] Method for Judging Inflammatory Diseases

According to the method of the present invention, the gene polymorphisms in the galectin-2 gene associated with inflammatory diseases, more particularly single nucleotide polymorphisms (SNPs), are detected to judge the onset of inflammatory diseases or to judge a possibility of onset of inflammatory diseases.

In the present invention, the phrase “detecting at least one gene polymorphism (e.g., a single nucleotide polymorphism) in the galectin-2 gene” refers to: (i) direct detection of the gene polymorphism (referred to as “a polymorphism in the gene”); and (ii) detection of a gene polymorphism in the complementary sequence of the aforementioned gene (referred to as “a polymorphism in the complementary sequence”) and deduction of the existence of the polymorphism in the gene based on the results of such detection. Since the nucleotides of the gene are not always completely complementary to the nucleotides of the complementary sequence, it is preferable to directly detect the polymorphisms in the gene.

A specific example of a preferable gene polymorphism in the galectin-2 gene is the C/T polymorphism at nucleotide 3279 in the nucleotide sequence of intron 1 of the galectin-2 gene as shown in SEQ ID NO: 1.

In the present specification, the nucleotide 3279 in the nucleotide sequence of intron 1 of the galectin-2 gene as shown in SEQ ID NO: 1 is equivalent to the nucleotide 3449 in the genomic sequence of the galectin-2 gene as shown in SEQ ID NO: 2.

As shown in Table 1 below, for example, when the nucleotide 3279 in the nucleotide sequence of intron 1 of the galectin-2 gene is C (3279C in intron 1 of galectin-2), it can be determined that inflammatory disease has been developed (onset) or is highly likely to be developed (onset).

On the other hand, when the nucleotide 3279 in the nucleotide sequence of intron 1 of the galectin-2 gene as shown in SEQ ID NO: 1 is T (3279T in intron 1 of galectin-2), it can be determined that inflammatory disease has not been developed or is less likely to be developed.

In the present description, the term “diagnosis” applied to diseases refers to the determination of the onset of diseases, the evaluation of a possibility of onset of diseases (a prediction of morbidity), elucidation of genetic factors of diseases, and the like.

The “judgment” of diseases can be carried out based on the results of the aforementioned method for detecting single nucleotide polymorphisms in combination with another form of polymorphism analysis (VNTR or RFLP) and/or the results of other test, according to need.

In the present specification, the term “inflammatory diseases” is not particularly limited, as long as induction of cell-adhesion factors or cytokines, which are known to be correlated with the inflammatory conditions, is observed. Examples of such diseases include chronic rheumatism, systemic lupus erythematodes, inflammatory enteritis, various allergy reactions, bacterial shock, and arteriosclerotic diseases such as myocardial infarction or cerebral apoplexy. A specific example thereof is myocardial infarction.

(Target of Detection)

The target of gene polymorphism detection is preferably genomic DNA. According to the circumstances (i.e., when the sequences of a polymorphism site and the region in the vicinity thereof are identical or completely complementary to the genome), cDNA or mRNA can be used. Such target can be obtained from samples, such as any biological samples: for example, body fluid such as blood, bone marrow fluid, semen, peritoneal fluid, or urine; tissue cells from the liver; and body hair such as hair. Genomic DNA and the like can be extracted from such samples and purified in accordance with a conventional technique.

(Amplification)

In order to detect a gene polymorphism, a region containing a gene polymorphism is first amplified. Amplification is carried out via, for example, PCR. It can also be carried out via other conventional amplification techniques, such as the NASBA method, the LCR method, the SDA method, or the LAMP method.

Primers are selected so as to be capable of amplifying, for example, a sequence consisting of at least 10 continuous nucleotides, preferably 10 to 100 nucleotides, and more preferably 10 to 50 nucleotides, containing the aforementioned site of single nucleotide polymorphism in the sequence as shown in SEQ ID NO: 1 (or SEQ ID NO: 3) and/or a complementary sequence thereof.

Primers may contain 1 or more substitution, deletion, or addition in such sequence as long as they can function as primers for amplifying a sequence consisting of a given number of nucleotides including the aforementioned SNP site.

The primer for amplification may be selected in such a way that either one of the forward and reverse primers hybridize to the SNP site so that amplification is carried out only when the sample has a single allele. Primers can be labeled with a fluorescent or radioactive substance, according to need.

(Detection of Gene Polymorphism)

The gene polymorphism can be detected via hybridization with a probe specific to one allele. Probes may be labeled with an adequate means such as a fluorescent or radioactive substance, according to need. Probes are not particularly limited as long as they contain the aforementioned SNP site, hybridize to the test material, and impart detectable specificity under the detection conditions to be employed. An oligonucleotide that can hybridize to a sequence consisting of at least 10 continuous nucleotides, preferably 10 to 100 nucleotides, and more preferably 10 to 50 nucleotides, including the aforementioned SNP site in the sequence as shown in SEQ ID NO: 1 (or SEQ ID NO: 3) or a complementary sequence thereof, can be employed as a probe. An oligonucleotide is preferably selected in such a way that the single nucleotide polymorphism is located in substantially the center of the probe. Such oligonucleotide may comprise one or more substitution, deletion, or addition in such sequence as long as it can function as a probe, i.e., it hybridizes to the sequence having the target allele but it does not hybridize to a sequence having another allele. Also, a probe may satisfy the aforementioned requirement by annealing to the genomic DNA to form a cyclic structure, as with the case of a single-strand probe (padlock probe) used for RCA (rolling circle amplification) amplification.

The hybridization conditions employed in the present invention are those sufficient for distinguishing alleles. An example of such conditions is stringent conditions where hybridization takes place when a sample has a single allele and does not take place when a sample has another allele. Examples of “stringent conditions” include those described in the Molecular Cloning: A Laboratory Manual, vol. 2 (Sambrook et al., 1989). Under stringent conditions, for example, hybridization takes place in a solution containing 6×SSC (the composition of 1×SSC: 0.15M NaCl, 0.015M sodium citrate, pH 7.0), 0.5% SDS, 5× Denhart's solution, and 100 mg/ml of herring sperm DNA while being incubated with a probe at 65° C. overnight.

A probe can also be used as a DNA chip by immobilizing one end thereof on a substrate. In such a case, a DNA chip may have immobilized thereon only a probe corresponding to a single allele or a probe corresponding to both alleles.

The gene polymorphism can also be detected via analysis of restriction fragment length polymorphism (RFLP). According to this technique, the sample nucleic acid is digested with a restriction enzyme, where whether or not it should be cleaved with a restriction enzyme depends on the genotype at the SNP site. Then, the size of the digested fragment is inspected to determine whether or not the sample nucleic acid was cleaved with the restriction enzyme. Thus, the polymorphisms of the sample are analyzed.

The gene polymorphisms may be detected by directly sequencing the amplification product (a method of direct sequencing). Sequencing can be carried out via conventional techniques, such as the dideoxy method or the Maxam-Gilbert method.

The gene polymorphisms can be detected via, for example, denaturing gradient gel electrophoresis (DGGE), single strand conformation polymorphism (SSCP), allele-specific PCR, hybridization utilizing allele-specific oligonucleotides (ASO), chemical cleavage of mismatches (CCM), the heteroduplex method (HET), primer extension (PEX), or rolling circle amplification (RCA).

[2] Kit for Diagnosing Inflammatory Diseases

The aforementioned oligonucleotide primer or probe can be provided as a kit for diagnosing inflammatory diseases which comprises such oligonucleotide. This kit may comprise a restriction enzyme, polymerase, nucleoside triphosphate, a label, a buffer, and the like, that are used for the method for analyzing gene polymorphisms.

[3] Method for Analyzing the State of Galectin-2 Expression

According to the present invention, the state of galectin-2 expression can be analyzed by detecting the aforementioned single nucleotide polymorphisms.

When the nucleotide 3279 in the nucleotide sequence of intron 1 of the galectin-2 gene as shown in SEQ ID NO: 1 is C (3279C in intron 1 of galectin-2), for example, the expression level of galectin-2 can be determined to be low. On the other hand, when the nucleotide 3279 in the nucleotide sequence of intron 1 of the galectin-2 gene as shown in SEQ ID NO: 1 is T (3279T in intron 1 of galectin-2), the expression level of galectin-2 can be determined to be high.

[4] Method for Screening for Therapeutic Agent for Inflammatory Disease

According to the present invention, a therapeutic agent for inflammatory diseases can be screened for by analyzing the expression level of the galectin-2 or galectin-1 genes in cells in the presence of a candidate substance and selecting a substance that alters such expression level. For example, the expression level of the galectin-2 or galectin-1 genes in cells is analyzed in the presence of a candidate substance, and a substance that increases or decreases such expression level can be selected. Particularly preferably, a substance that increases such expression level can be selected. According to the present invention, the binding between lymphotoxin-α (LTA) and the gene product of galectin-2 or galectin-1 is assayed in the presence of a candidate substance, and a substance that inhibits such binding is selected. Thus, a therapeutic agent for inflammatory diseases can be screened for.

For example, such screening can be carried out by a step of bringing cells into contact with a candidate substance, a step of analyzing the expression level of the galectin-2 or galectin-1 genes in the cells, and a step of selecting a candidate substance that can alter the expression level of such genes, as compared with the conditions that pertain in the absence of the candidate substance, as a therapeutic agent for inflammatory diseases.

Any substance can be employed as a candidate substance. The type of candidate substance is not particularly limited, and an individual low-molecular-weight synthetic compound, a compound existing in an extract of a naturally occurring substance, a compound library, a phage display library, or a combinatorial library may be used. Preferably, a candidate substance is a low-molecular-weight compound, and a compound library of low-molecular-weight compounds is also preferable. A compound library can be constructed by a method known in the art. A commercially available compound library can also be used.

[5] Method for Assaying transcriptional Activity of Galectin-2

According to the present invention, a galectin-2 gene fragment containing the aforementioned single nucleotide polymorphism is introduced into cells, the cells are cultured, and the expression of such gene is analyzed. Thus, transcriptional activity of galectin-2 can be assayed.

According to a preferred embodiment of the present invention, a transcription unit comprising a reporter gene ligated to a site downstream of the aforementioned galectin-2 gene fragment is introduced into cells, such cells are cultured, and reporter activity is assayed to analyze the expression of such gene.

When SNP is present at the promoter site, for example, cells where a system containing a reporter gene ligated to a site downstream of the gene containing such SNP has been introduced are cultured and the reporter activity is assayed. Thus, differences in transcription efficiency resulting from SNP can be assayed.

Examples of the reporter genes used herein include luciferase, chloramphenicol, acetyltransferase, arid galactosidase genes.

[6] Method for Screening for Substance that Inhibits or Promotes Transcriptional Activity of Galectin-2

According to the present invention, the galectin-2 gene fragment containing the aforementioned SNP is introduced into cells, such cells are cultured in the presence of a candidate substance that inhibits or promotes transcriptional activity of galectin-2, and the expression of such gene is analyzed. Thus, a substance that inhibits or promotes transcriptional activity of galectin-2 can be screened for.

According to a preferred embodiment of the present invention, a transcription unit comprising a reporter gene ligated to a site downstream of the aforementioned galectin-2 gene fragment is introduced into cells, such cells are cultured, and reporter activity is assayed to analyze the expression of such gene.

For example, cells having a system wherein the reporter genes are ligated to a site downstream of the genes having single nucleotide polymorphisms (e.g., 3279T in intron 1 of galectin-2), in which the expression level of galectin-2 is significantly high, are cultured in the presence of and in the absence of a candidate substance. If the reporter activity decreases when the culture is conducted in the presence of a candidate compound, this candidate compound can be selected as a substance that inhibits transcriptional activity of galectin-2.

The reporter genes as mentioned above can be employed.

Any substance can be employed as a candidate substance. The type of candidate substance is not particularly limited, and an individual low-molecular-weight synthetic compound, a compound existing in an extract of a naturally occurring substance, a compound library, a phage display library, or a combinatorial library may be used. Preferably, a candidate substance is a low-motecular-weight compound, and a compound library of low-molecular-weight compounds is also preferable. A compound library can be constructed by a method known in the art. A commercially available compound library can also be used.

The present invention also includes a substance that inhibits or promotes transcriptional activity of galectin-2 which is obtained by the screening method of the present invention. Such substance that inhibits or promotes transcriptional activity of galectin-2 is useful as a candidate substance of various agents, such as a therapeutic agent for myocardial infarction, an anti-inflammatory agent, and an immunosuppressive agent.

[7] Method for Screening for Transcription-Regulatory Factor of Galectin-2

According to the present invention, the SNP-containing gene fragment is brought into contact with a sample which is deduced to contain a transcription-regulatory factor of galectin-2, and binding between such fragment and the transcription-regulatory factor is detected. Thus, a transcription-regulatory factor of galectin-2 can be screened for. Binding between the SNP-containing gene fragment and the sample which is deduced to contain a transcription-regulatory factor of galectin-2, is detected via gel-shift analysis (electrophoretic mobility shift assay (EMSA)), DNase I footprinting, or the like, with gel-shift assay being preferable. In gel-shift assay, molecular size is enlarged upon binding of proteins (transcription-regulatory factors), and this results in lowered mobility of DNA in electrophoresis. Accordingly, a ³²P-labeled gene fragment is mixed with a transcription-regulatory factor and the resultant is subjected to gel electrophoresis. If the position of DNA is observed via autoradiography, DNA to which a transcription-regulatory factor has been bound is found to move slowly. Thus, it is detected as a band that moves more slowly than usual bands.

Hereafter, the present invention is described in greater detail with reference to the following examples, although the present invention is not limited to these examples.

### EXAMPLES

(A) Methods and Materials

(1) E. coli Two-Hybrid System

The BacterioMatch™ Two-Hybrid System library construction kit (Stratagene) was used. Cultured human coronary artery smooth muscle cells (HCASMC) for library construction were purchased from BioWhittaker. mRNA was prepared from 1×10⁷ cells using the FastTrack 2.0 kit (Invitrogen), 5 μg of HCASMC mRNA was used to construct a cDNA library in accordance with the protocol of the kit, and two-hybrid screening was carried out in accordance with the protocol.

(2) Preparation of Recombinant Galectin-1, Galectin-2, Galectin-3, and LTA and Observation of Binding between LTA and Galectin via Immunoprecipitation

Recombinants of full-length galectin-1, galectin-2, and galectin-3 were prepared using the pET 28 vector system (Novagen), and they were allowed to express in E. coli in accordance with the protocol of the kit, followed by purification. Recombinant LTA was prepared using the pET29 system (Novagen). The anti-LTA antibody (R&D Systems) was allowed to crosslink to the HiTrap NHS-activated HP sepharose (Amersham) in accordance with the protocol of the kit (anti-LTA antibody sepharose). The LTA-galectin binding experiment was carried out by adding 5 μg of galectin-1, galectin-2, or galectin-3 to 10 ml of binding buffer (10 mM Tris/HCl, pH 7.5, 150 mM NaCl) and further agitating the mixture for 1 hour. The resultant was subjected to centrifugation at 1,600 rpm for 10 minutes, the supernatant was discarded, the precipitate was washed 3 times with a wash buffer (10 mM Tris/HCl, pH 7.5, 150 mM NaCl, 0.1% NP-40), the resultant was lysed using 50 μl of 5×SDS-sample buffer (125 mM Tris/HCl, 4% SDS, 20% glycerol, 10% beta-mercaptoethanol, 0.04% bromophenol blue, pH 6.8), so as to obtain the sample.

After SDS polyacrylamide gel electrophoresis, the sample was transferred onto a nitrocellulose membrane, and signals were detected using the anti-T7, antibody (Novagen) by the ECL method (Amersham). In order to observe the interaction (binding) between LTA and galectin-2 in a forced expression system utilizing COS7 cells (Riken Cell Bank), recombinant LTA was introduced into the pFLAG-CMV-5a vector (Cosmo Bio Co., Ltd.), recombinant galectin-2 was introduced into the pCMV-Myc vector (Clontech), and the resultants were transfected into the COS7 cells using the FuGene reagent (Roche). The cells were recovered 36 hours later, and proteins were extracted using a buffer for cell protein extraction (10 mM Tris/HCl, 150 mM NaCl, 0.5% NP-40). In order to suppress nonspecific adsorption, 50 μl of protein A sepharose (Amersham) was added to the extract, the mixture was agitated for 1 hour, the agitated mixture was centrifuged at 1,600 rpm for 10 minutes, and the supernatant was used as the sample for immunoprecipitation. The anti-LTA antibody (5 μg) was added to the sample, the mixture was agitated at 17° C. for 1 hour, 50 μl of protein A sepharose was added thereto, the resultant was agitated at 17° C. for 1 hour, centrifugation was carried out at 1,600 rpm for 10 minutes, the precipitate was washed 3 times with a wash buffer (10 mM Tris/HCl, pH 7.5, 150 mM NaCl, 0.1% NP-40), so as to obtain the sample. After SDS-PAGE, the sample was transferred onto a nitrocellulose membrane, and signals were detected using the anti-Myc-tag antibody (Santa Cruz) and the anti-FLAG-tag antibody (Sigma).

(3) Analysis of Correlation between SNP in Galectin-2 Gene and Myocardial Infarction

The group of myocardial infarction patients and the control group, the method for sampling DNAs from these groups, DNA sequencing, DNA typing, and the statistical correlation analysis were determined in accordance with conventional techniques (Ozaki, K. et al., Nature Genetics 32, 650-654, 2002). SNPs in the galectin-1 and galectin-2 genes were identified and discovered via PCR direct sequencing with the use of DNAs obtained from 16 myocardial infarction patients and from 16 controls.

(4) Luciferase Assays

A region between nucleotide 3188 and nucleotide 3404 of the galectin-2 gene containing SNP (3279C or T) in intron 1 was amplified via PCR using a genomic DNA template, and the amplification product was cloned into a site downstream of luciferase of the galectin-2 promoter-pGL3-enhancer vector. These plasmids (2 μg) and 100 mg of pRL-TK vector (the internal standard vector for adjusting transfection efficiency, Promega) were transfected into HeLa cells (JCRB9004, Human Science Research Resources Bank) and into HepG2 cells using the FuGene reagent. The cells were collected 24 hours later, and luciferase activity was assayed.

(5) Tandem Affinity Purification

Tandem affinity purification was carried out in accordance with the method of Rigaut, G. et al. (Nature Biotechnology, 17, 1030-1032, 1999). A fusion cassette encoding His tag, TEV cleavage site, and S tag as a TAP-tag sequences was constructed in the pCMV-Myc vector (Sigma). This TAP vector allows the expression of the carboxy-terminal TAP-tagged and amino-terminal Myc-tagged target proteins in mammalian cells under the control of the cytomegarovirus promoter. The TAP vector was transiently transfected into HeLa cells (JCRB9004, Human Science Research Resources Bank). The band of the target protein was analyzed using the MALDI-TOF mass spectrometer (APRO Life Science).

(6) Coimmunoprecipitation Experiment

The coimmunoprecipitation experiment was conducted in the following manner using endogenous α-tubulin and FLAG-tagged galectin-2 or LTA. The FLAG- or S-tagged LTA, galectin-2, and lacZ (a negative control) were transfected into HeLa cells using the Fugene. Immunoprecipitation was carried out in a lysis buffer (20 mM Tris pH 7.5, 150 mM NaCl, 0.1% Nonident P-40). Cells were lysed 24 hours after the transfection, and immunoprecipitation was conducted using the anti-FLAG tag M2 agarose (Sigma). The immune complex was visualized using the HRP-conjugated S-protein (Novagen), the anti-FLAG M2 peroxidase conjugate (Sigma), or a mouse monoclonal antibody (Molecular Probes) against human α-tubulin, and the HRP-conjugated anti-mouse IgG antibody.

(7) Confocal Microscopy

The polyclonal anti-human galectin-2 antisera were raised in rabbits using recombinant proteins synthesized in E. coli. The antisera were subjected to Western blotting, and they were found to exhibit no cross-reactivity to galectin-1 or galectin-3 having similar structures. The polyclonal anti-galectin-2 antisera and goat anti-human LTA IgG (R & D System) or the mouse anti-human α tubulin monoclonal IgM antibody were used with the Alexa secondary antibodies (molecular probes). U937 cells (JCRB9021, Human Science Research Resources Bank) were stimulated for 30 minutes with phorbol myristate acetate (PMA, 20 ng/ml) and then immobilized. Thereafter, the resultant was incubated with the corresponding primary antibodies and then with the corresponding Alexa secondary antibodies in phosphate-buffered physiological saline containing 3% bovine serum albumin.

(8) Immunohistochemical Analysis

Tissue samples were obtained from 16 myocardial infarction patients via directional atherectomy. Immunohistochemical analysis was carried out using goat anti-human LTA IgG (R&D Systems) and rabbit polyclonal anti-human galectin-2 antibodies in accordance with conventional techniques (Minami, M; et al., Arterioscler. Thromb. Vasc. Biol. 21, 1796-1800, 2001; and Shi, S. R., et al., Hum. Mutat. 15, 7-12, 2000). Staining of adjacent slices was carried out using human-cell-type-specific monclonal antibodies against SMC-2 actin and CD68 (DAKO). For double-label immunohistochemical analysis, slices were incubated with the anti-LTA antibodies, biotinylated swine anti-goat IgG, and the avidin-biotin-peroxidase conjugate in that order, followed by visualization with 3,3′-diaminobenzidine tetrahydrochloride (Vector Labs). Subsequently, the slices were incubated with the rabbit polyclonal anti-human galectin-2 antibodies and then with alkaline phosphatase-conjugated swine anti-rabbit IgG, followed by visualization with the 5-bromo-4-chloro-3-indoxyl phosphate and nitroblue tetrazolium chloride (BCIP/NBT) substrate system.

(B) Results

(1) Identification of Protein Binding to LTA, the Myocardial Infarction Susceptibility Gene Product (Screening)

In order to screen for a novel protein binding to LTA, the E. coli two-hybrid system was used, and galectin-1 was identified as a candidate protein binding to LTA from the two-hybrid library derived from blood vessel smooth muscle cells.

(2) Observation of Binding Between LTA and Galectin-1 or Galectin-2 In Vitro

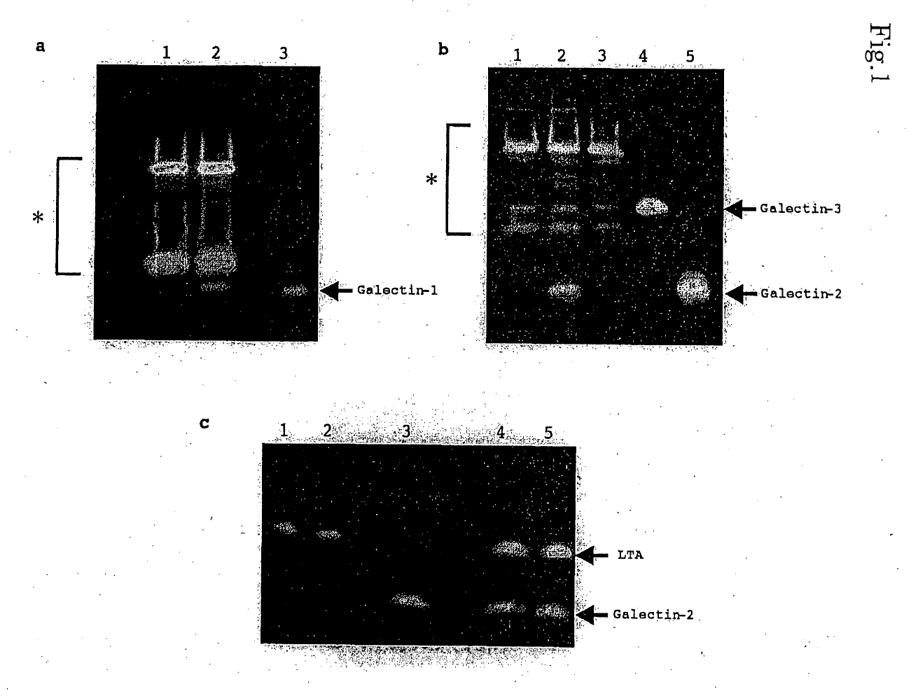

Recombinant galectin-1 (T7-tagged at the N-terminus) and LTA were separately expressed in E. coli, purified, allowed to react with anti-LTA antibody-crosslinked sepharose, washed, and then subjected to SDA-PAGE. Galectin-1 was detected via Western blotting using the anti-T7 antibody (FIG. 1a).

In FIG. 1a, in lane 1, galectin-1 was subjected to immunoprecipitation using anti-LTA antibody sepharose (a negative control). In lane 2, Galectin-1 was incubated with LTA, and the conjugate was subjected to immunoprecipitation using anti-LTA antibody sepharose. In lane 3, recombinant galectin-1 (100 ng) was used as a positive control. Asterisks indicate nonspecific bands derived from the immunoglobulin (Ig) heavy chain and light chain in anti-LTA antibody sepharose.

Recombinant proteins of galectin-2 and galectin-3 which have high homology to galectin-1 were prepared from E. coli, and the binding thereof to LTA was analyzed in the same manner as described above. As a result, galectin-2 was also found to bind to LTA (FIG. 1b).

In FIG. 1b, galectin that had been coimmunoprecipitated with LTA was detected via Western blotting using the anti-T7 tag monoclonal antibody and horseradish peroxidase conjugate anti-mouse IgG. In lane 1, galectin-3 was incubated with LTA and the conjugate was immunoprecipitated using anti-LTA antibody sepharose. In lane 2, galectin-2 was incubated with LTA and the conjugate was immunoprecipitated using anti-LTA antibody sepharose. In lane 3, galectin-2 was immunoprecipitated using anti-LTA antibody sepharose (a negative control). In lanes 4 and 5, 100 ng of galectin-3 (lane 4) or galectin-2 (lane 5) was used as a positive control. Asterisks indicate nonspecific bands derived from the immunoglobulin (Ig) heavy chain and light chain in anti-LTA antibody sepharose.

Further, galectin-2 was subjected to forced expression with LTA Thr26 and LTA Asn26 (Ozaki, K. et al., Nature Genetics 32, 650-654, 2002) in COS7 cells (monkey kidney cell lines), and binding was confirmed at the cell culture level (FIG. 1c).

FIG. 1C shows the results of coimmunoprecipitation of LTA and galectin-2 using the anti-LTA antibody. The Myc-tagged galectin-2 plasmid or FLAG-tagged LTA plasmid (Thr26 or Asn26) was transfected into COS7 cells, and a lysate was prepared and subjected to immunoprecipitation with protein A sepharose and the anti-LTA antibody. The galectin-2 coprecipitated with LTA was detected via Western blotting using an Myc (galectin-2) or FLAG (LTA)-anti-monoclonal antibody-horseradish peroxidase conjugate. In lanes 1 and 2, LTA Thr26 (lane 1) or LTA Asn26 (lane 2) was transfected for precipitation (a positive control for LTA). In lane 3, galectin-2 was transfected for precipitation (a positive control for galectin-2). In lanes 4 and 5, galectin-2 was transfected with LTA Thr26 (lane 4) or LTA Asn26 (lane 5) for coprecipitation.

(3) Correlation between Single Nucleotide Polymorphisms in Galectin-2 Gene and Myocardial Infarction

Galectin-1 and galectin-2 were found to bind to LTA, and functional variations in these gene products were found to have led to functional variations in LTA, which could be associated with susceptibility to myocardial infarction. Accordingly, novel single nucleotide polymorphisms (SNPs) in these genes were identified and discovered, and the discovered SNPs were used to subject about 2300 patients and about 2300 controls to the case-control association study. As a result, it was found that the quantity of minor homozygots (TT allele) of the novel SNPs (3279 C>T) in intron 1 of the galectin-2 gene was significantly small in myocardial infarction patients (χ²=25.3, P=0.0000005; odds ratio=1.6) (Table 1) (where the nucleotide number depends on the variant designation, Dunnen J. T. et al., Hum. Mutation 15, 7-12, 2000). This indicates that SNPs at nucleotide 3279 in intron 1 of galectin-2 are factors that act protectively in myocardial infarction and that functional variations in galectin-2 may be associated with myocardial infarction.

Table 1: Correlation between Myocardial Infarction and SNP in Galectin-2

| | | | | | χ² [P value] (Odds ratio) <95% CI> | | | |
|---|---|---|---|---|---|---|---|---|
| Genotype Galectin-2 | | | | | Genotype | Allele | CC vs | TT vs |
| intron 1 3279C > T* | MI | | Control | | frequency | frequency | Others | Others |
| CC | 1047 | (46.8%) | 996 | (41.6%) | 29.6 | 25.5 | 12.8 | 25.3 |
| CT | 987 | (44.2%) | 1069 | (44.7%) | [0.00000038] | [0.00000044] | [0.00034] | [0.00000050] |
| TT | 202 | (9.0%) | 329 | (13.7%) | | (1.71) | (1.24) | (1.60) |
| Total | 2236 | (100%) | 2394 | (100%) | | <1.41-2.08> | <1.10-1.39> | <1.33-1.93> |

The nucleotide sequence in the vicinity of a novel SNP (3279 C>T) in intron 1 of the galectin-2 gene that was identified in the present invention is shown below (SEQ ID NO: 3, “Y” represents “C” or “T” in SEQ ID NO: 3).

5′-CCCCCCCAGCTCTAGGGACGACCACACCCCCACCCAGTTCTGCCTGT
CTCTCTCTGCGCCTTTGACTCTGTTGGGTGGGGACAAGGCTCCCGGGCCT
GCACCCTCCCGCAGCTCTCAGCATCCCTATTTGTCCAAGTGCACCCCTGA
CCCTGGACTTCCGAGTGCTTCTGCCCTGCAGCAGCCCCCACCTCTATCCT
TGGGGTTTGAGCTTTGCTGTTTCAGTCAGGCAGCCCCCAGGAGCTGCAAG
GGGAGTGTGGGTGCTTCTCTTAGTCCAGGCCCAGCTCCCCTATCCTGGCC
TGACTGTTGCAGGGCTCGGGGTGTGGGCACAGGCTGCTGGCAGGAGGCAG
GGAGCCATCTCCTGATGCTTGGTGTTAGA[C/T]GTGTGTGTGCGCAGGG
CACACGTCTGTGAGTGTCTGTGTGGCGGGCACACCTGTCTTCTGTTTCTT
GTTTGAGCCCCTTTTGGACTGTCCTCACTGGATAACCTCATCTCCCAGAG
ATAATGGTCTTTGTCAGTGAGAGACTGATTTTTTTTTTTTTTTTTTTTTT
TTTGAGACGGAGTCT-3′

“C/T” represents the SNP (3279 C>T) in intron 1. The underlined CTGCGCCTTTGACTCTGTT and TCTTTGTCAGTGAGAGACTG represent PCR primers, and the underlined CCTATCCTGGCCTGACTGTT represents a sequence primer.

(4) Influence of SNPs at Nucleotide 3279 in Intron 1 of Galectin-2 Gene on Transcriptional Activity of Galectin-2 Gene

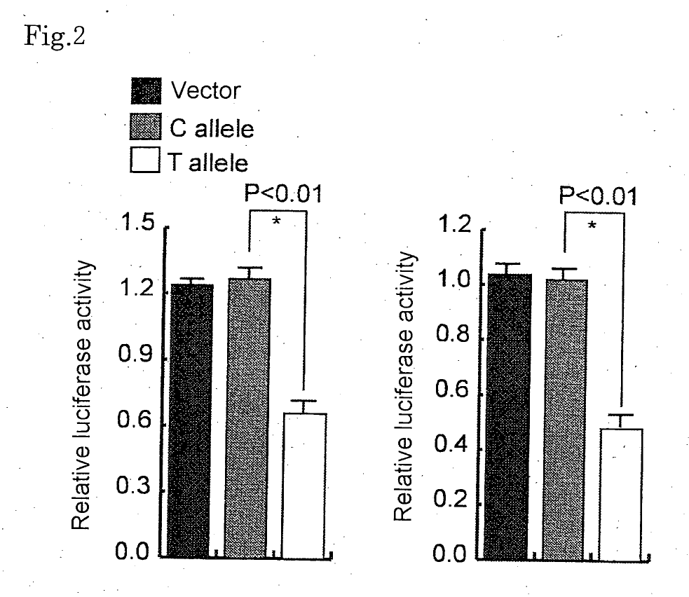

In order to assay the influence of SNP at nucleotide 3279 in intron 1 on transcriptional activity of the galectin-2 gene, reporter gene assays (luciferase assays) were carried out. A DNA fragment composed of a region between nucleotide 3188 and nucleotide 3404 in intron 1 of the galectin-2 gene was cloned into a site downstream of the SV40 enhancer of the pGL3 enhancer vector in the 5′ to 3′ orientation to prepare a reporter vector. The reporter vector was transfected into HeLa cells and in HepG2 cells, and luciferase activity was assayed 24 hours later using the Dual-Luciferase Reporter Assay System (Promega). The results are shown in FIG. 2 (left column: the results in HeLa cells; right column: the results in HepG2 cells).

(5) Interaction between Galectin-2 and Microtubule

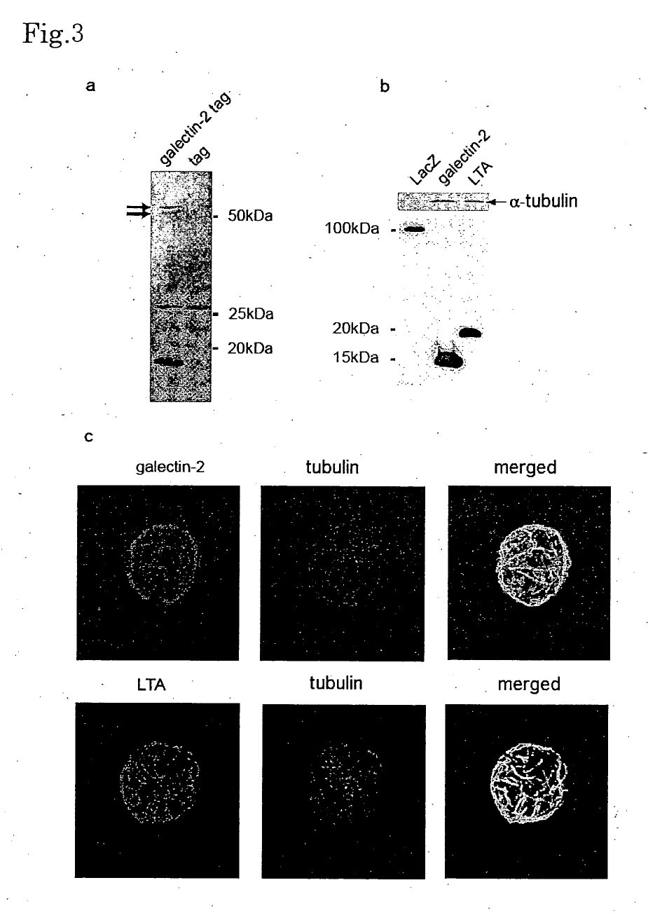

In order to investigate the LTA secretion regulatory mechanism by galectin-2, intracellular molecules that interact with galectin-2 were searched for using a tandem affinity purification (TAP) system. As a result, two specific bands, which can be detected only when the galectin-2-TAP tag was expressed, were identified (FIG. 3a). Through a MALDI-TOF mass spectrometry analysis, these two bands were found to correspond to α- and β-tubulins, which are important components of microtubules. With the use of HeLa cells transfected with a plasmid to express FLAG-tagged galectin-2, coimmunoprecipitation of endogenous tubulins with galectin-2 was confirmed (FIG. 3b). Tubulins were also coimmunoprecipitated with LTA (FIG. 3b). The image obtained via confocal microscopy of dually immunostained U937 cells revealed that galectin-2 and α-tubulin were colocalized as reticular filamentous networks developed in the cytoplasm (FIG. 3c). This indicates that galectin-2 may be associated with intracellular trafficking.

(6) Expression and Colocalization of Galectin-2 and LTA in Sample Obtained via Coronary Atherectomy

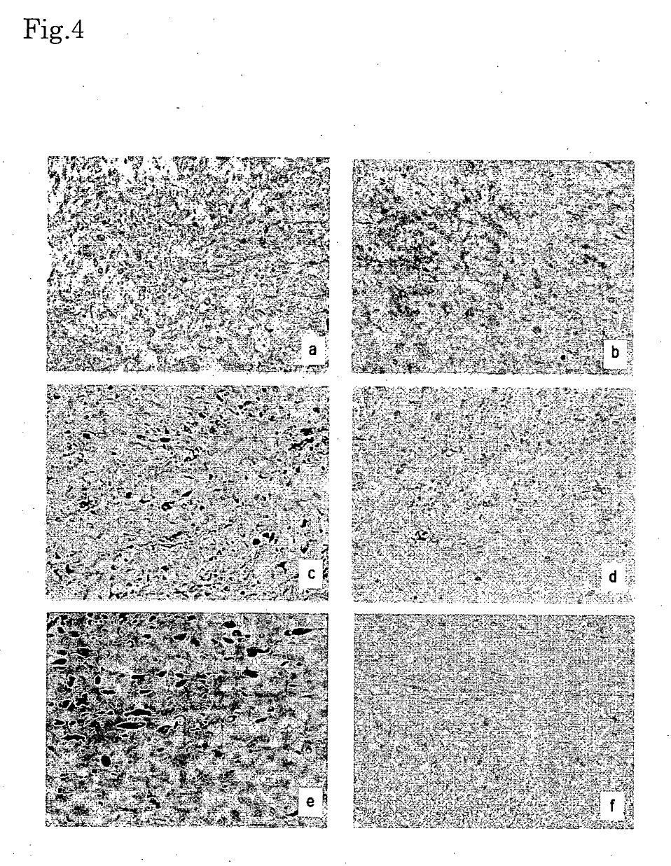

To examine whether or not galectin-2 and LTA are expressed in a lesion of myocardial infarction (i.e., the atherosclerotic lesion of the coronary artery) and to inspect the sites of expression when they are expressed, the samples obtained via coronary atherectomy were subjected to immunohistochemical analysis using anti-LTA or anti-galectin-2 antibodies. As shown in FIG. 4a and 4b, immunoreactivities for both LTA and galectin-2 were detected in intimal cells in atherosclerotic plaques, some of which were spindle-shaped or contained vacuolated, round cytoplasms. Immunostaining of adjacent slices with anti-smooth muscle cell (SMC) α-actin or anti-CD68 revealed that the majority of these cells were either SMCs or SMC-derived foam cells, and macrophages were occasionally observed (FIG. 4c and d). Co-expression of LTA and galectin-2 was observed in the majority of polymorphic SMCs via double-label immunohistochemical analysis (FIG. 4e). In contrast, no protein expression was observed in atrophic SMCs of fibrous plaques with scanty cellurarity or in normal medial SMCs (FIG. 4f). These results indicate that LTA and galectin-2 are coexpressed in SMCs and macrophages in the intima of human atherosclerotic plaques but are absent in quiescent or normal medial SMCs.

### INDUSTRIAL APPLICABILITY

According to the present invention, novel single nucleotide polymorphism (SNP) associated with the onset and development of inflammatory diseases such as myocardial infarction was identified. Use of the SNP identified by the present invention enables the establishment of a method for diagnosing inflammatory diseases such as myocardial infarction or a method for developing a therapeutic agent for inflammatory diseases.

1. A method for judging inflammatory diseases which comprises detecting at least one gene polymorphism in the galectin-2 gene.

2. The method of claim 1 which comprises detecting at least one single nucleotide polymorphism in the galectin-2 gene.

3. The method of claim 2 which comprises detecting the C/T polymorphism at nucleotide 3279 in the nucleotide sequence of intron 1 of the galectin-2 gene as shown in SEQ ID NO: 1.

4. The method of claim 1 wherein the inflammatory disease is myocardial infarction.

5. An oligonucleotide that can hybridize to a sequence consisting of at least 10 continuous nucleotides including the nucleotide 3279 in the nucleotide sequence of intron 1 of the galectin-2 gene as shown in SEQ ID NO: 1 or a complementary sequence thereof, and that can be used as a probe in the method of claim 1.

6. An oligonucleotide that can amplify a sequence consisting of at least 10 continuous nucleotides including the nucleotide 3279 in the nucleotide sequence of intron 1 of the galectin-2 gene as shown in SEQ ID NO: 1 and/or a complementary sequence thereof and that can be used as a primer in the method of claim 1.

7. The oligonucleotide of claim 6 wherein the primer is a forward and/or reverse primer.

8. A diagnostic kit for inflammatory diseases which comprises at least 1 oligonucleotide according to claim 5.

9. The kit of claim 8 wherein the inflammatory disease is myocardial infarction.

10. A method for analyzing the state of galectin-2 expression which comprises detecting the C/T polymorphism at nucleotide 3279 in the nucleotide sequence of intron 1 of the galectin-2 gene as shown in SEQ ID NO: 1.

11. A method for screening for a therapeutic agent for inflammatory diseases which comprises steps of analyzing the expression level of the galectin-2 or galectin-1 genes in cells in the presence of a candidate substance and selecting a substance that alters such expression level.

12. A method for screening for a therapeutic agent for inflammatory diseases which comprises steps of analyzing the expression level of the galectin-2 or galectin-1 genes in cells in the presence of a candidate substance and selecting a substance that increases such expression level.

13. A method for screening for a therapeutic agent for inflammatory diseases which comprises steps of assaying the binding between lymphotoxin-α (LTA) and the gene product of galectin-2 or galectin-1 in the presence of a candidate substance and selecting a substance that inhibits such binding.


SEQUENCE LISTING (6 sequences)

SEQ ID NO: 1 - DNA, 7967 bp, Homo sapiens
gtgaggacactagccccctgctgcctgccccactctgttcatctttgtctttgcctgggtgggggcttttagggaaaaccattgctgtccctctctgggcctcagtttccccatctgtgcagcaaagaagttggacagaggtctttttttaaaaaacagcatcttgggccaggcgtggtggctcctgcttgtaatcccagcactttgggaggccgaggctggtggatcatctgaggttgggagtttgagaccagcctgaccaacatggagaaaccccgtctctactaaaaaaatacaaaattggctaggcctggtggcacatgcctgtaatcccagctaatggggaggctgaggcaggagaatcacttggacctgggaggcagaggttatggtgagccgagattgtgccattgcactccagcctgggcaacaagagtgagactccatctcaaaacaacaacaacaatacagcatcttgctctgtcaccaggtggagtgcagtggtggcaatcataactcactacggacttgacctccttggcttaaatgatcctcccacctcagcctcttgagtagctgggaccccaggcactcactaccacactggctaattttgtttgtttcttttctttctctttttttttttttttttgagatggagtctcgctctgttgcccaggctggagtgcagtggcccgatctcagctcactgcaacctctgctgcctgggttcaagcaattctctggtctcagcctcccaagtagctgggattacaggtatgtgtcaccacacctggctaatttttttttttttgttgagatggagtttctgttgcccaggctggagtgcaatggcacgatctcggctcaccacaacttccacctcccaggttcaagcgattctcctgcctcagcctcctgagtagcagggattacaggcatgggccaccacacccgattaattttgtatttttagtagagatggggtttctccatgttggtcaggctggtcttgaactcctgatctcaggtgatccacctgccttggcctcccaaagtgctgggattacaggtgtgagccactgctcctggcctaatttttgtatttttaaagtagagacagggtttcaccatgttggtcaggctgatctcgaactcctgacctcaggtgatccgcccaccttggcctcccaaagtgctgggattacaggtgtgagccaccccacccagcttatttcttatttttcgtagagatgaggtctcactatgttgctcaggctgatatcaaactcctgggttcaagggatcctcctgccttggcctctcgaagtgctgagattacaggtgtgagccactgtgcctggcctccattgatctttatagagataaaaaaaaatctcagcttgggcaatatagtgagaccttttctgctacaggtgcatgccactacgctttgccttaaaaaattagtgggggtagcggcacactcctcagccttgggaggctgaggatcacttgagcccaggaggtcgaggctacagtgagccgtaattgcactactgtactccagcctgggcaacagagtgagaccttgtctcatatacccacacacaaaacccaagtcttggagagcaaattgcccaaggccacaagctgcaaatcacaaggggttgagtggattcccactgaggtctctgattcgttgattctacaccagactctgccacagctttactgtgtggccttggccaagtcactgaccgtctctgagccccagtcttccttacatctgtggaaggggatcacaggctgcctcttctgaggattagatggtgtattcattgcctagggctgcaataacaaattaccaccaaattgtgggtggcttcacacgatagacgtttgttctgtcttggttttggtgactagaaacctgaaaccaaggtgctacagggctacgctcctgctgaaggcgcaaggggagggttctttcttgcctcttccagcttctggtggctcctcgcattccttggcttgcatcactccaatctctgcctccaacttcacgtggactcctctgtgtgtctccgtctctgtgtctatatttctctcctcttatgagaacactggtcgtattggatttaggaccaaccctaaaccagtatgacctcttaactcgattacatatgcaaaggaactatttttaaataggtcacattgaggctgggcggggtggctcacccctgtaatcccagcactttgggaggccgaggcaggcggatcacttgagatcaggagttcaagaccagcctggccaacatggtgaaaccctgtctgtactgaaaatacaaaaacaaaaaagaagaagaagaagaaaaaaaaattagacagatgttgtggtgggcacccgtaatcccagctacttgggaggctgaggcaggagaatcggttgaacccgggaggcagaggttgcagtgagccgagatcgagccactgtactccagcctaggtgacagagtgagacttcatctcaaaaaaaaaaaaaaaggtcacattgacaggttccaggtggacatgaattttcgggggacgctattcaagtgcaggggggatgcaggatgtgaatgtgccaggggtcctgcgtggaagggtctatgccctcatcaccctctgcctctcggggaggactgctgtggcccacggactctccccaccttctctttcctggtcatctcacctctgccttttctttcctctctctccagctccagaggccatatcatccaaatcccttatacgacagataagggaaccaaggcccagaaaggggctaagctggccccaggcccctctgccaattaggggcagagtcggcactagagtctgggcccccaactccccacccccccagctctagggacgaccacacccccacccagttctgcctgtctctctctgcgcctttgactctgttgggtggggacaaggctcccgggcctgcaccctcccgcagctctcagcatccctatttgtccaagtgcacccctgaccctggacttccgagtgcttctgccctgcagcagcccccacctctatccttggggtttgagctttgctgtttcagtcaggcagcccccaggagctgcaaggggagtgtgggtgcttctcttagtccaggcccagctcccctatcctggcctgactgttgcagggctcggggtgtgggcacaggctgctggcaggaggcagggagccatctcctgatgcttggtgttagacgtgtgtgtgcgcagggcacacgtctgtgagtgtctgtgtggcgggcacacctgtcttctgtttcttgtttgagccccttttggactgtcctcactggataacctcatctcccagagataatggtctttgtcagtgagagactgatttttttttttttttttttttttttgagacggagtctcgctctgtcgcccaggctggagtgcagtggcgccatcttggctcactgcaagcaccgcctcccgggttcacgccattctcctgcctcagcctcccgagtagctgggactacaggcgcctgccaccacgcccggctaattttttgtatttttagtagagacagggtttcaccgtgttagccaggatgatctcactctcctgacctcgtgatccgcccccctcggcctcccaaagtgctgggattacaggtgtgagccaccgcccctggccagcaagagactgattttaatcccgtctgtctggctccaaaatctggacccaaccccgttgtgttaagcaaagacatggggagttaggtgtccagcctccaaaccccactttctctaaagcagggaggttttgctcccaggagacaacggaccctgtctggagacattcttggttgtcaccgctcaggggagggtgtcactgacatccagtgggtagaggccaggaatactgctcaacatcctacaacacaagagacagaccccaacaaagaaatgcctgccccaaacgtccagacggccaaggctgagaagctctggtctgagcagcctcctgtctgacatgccgccgtcatggcccgctgtcctgggttaagcattgctgcctcctccaggcgtctcttataaaatgtactgccaggccgggcacagtggcttacacctgtaatcccaacactttgggaggccgaggtgggaggatcctttgagctcaggaggtcgaggctggcctggacaacatagtgagaccccatctgaaaaaaaaaaaaatcagctgggccaggtgggatgcctgtggtccaggctacctggggggctgaggtgggaggattgcttgagcccaagaggtcaaggctgtagtgagctctgatcatatcactgcactctagcctgggtgacagagcaagacctttaaaaaaaaatgtattaccggctgaggcaggaggaccacttgagggtcaggagttccagagcagcctgggtaacatagcaaaaccctatctctacaaaaattttaaaaattagctgggcatggtggcacacgtctatagtcgtagctacttaggaggctgaggcaggagaatcgtttgagctcaggaggctatgaggctgcatgcagtgagttataatcgtgctactgcactccatcttgggtgacagagcaagaccctgtctcaaaaaaaaaaaaaaaagaaagaaaagaaaaaaaatgctgggtgtggtatctcacccctataatcccagaactttgggaggcccagaggggaggatcacttgaggtcaggagttcgagaccaacctggccaaagtggtgaaaccccgtctctactaaaaatacaaagaaaattagctagatatggtggtgggagcctgtaatctcagtgactcgggaggctgaggcaggataattgcttgaatctgggaagtggaggttgcagtgagccaagattgcaccactgcactccagcatgggtgacagagtgagactccatctcaacatctcaaaaaaaaaaaaaaaagaacttactgcctgtggaagagttgagcaatacctaacaacctacccctacatgtgaccaaccagcgggtcacttcctcctctgcagagaggaggcggctgccagcgagagggcactgagggtcctcccatggccactgcccccttgacttctggcaaagtgccccagtccaatgagctcattcagggcatctcagatcatgctttttctggaaataaaaagtcagtgagcagaactcccacaatgtaaaagtgtcctcccataagttgttctaaatctttggtgcctgttgcgtcctggtcagaccaaccctcaccctctggtcatagatgcgaaaactggtcttgggtaatgagtttttttttttttttttttttagacagagtcttgctctgtcgcctggcacaatctcggctcactgcaacctccacctcctgggttcaagcgattctcttgcctcagcctcccaagtagctgggacgacaggcatgtgccaccacacccggctaatttttgtatttttaatagagacagggtttctccatgttggccaggccagtctcaaactcctgacctcaggtgatccacccgactcagcctcccaaagtgctgggattacaggcgtgagccaccgctcccagccctgggtaatgagctttgaaaacccagcttagaaatcttccctagtaaccatcgtgaggctagaggaggctcctactgtacagaaattcaggtgctgctttcctatggaaaataaggagcagatgaatcttaacaacaagtaatcaaaatgatggtcatttgggcagaccactgtccagaaaaaaagaaaaaatttaaaaaagaaaattaaggctgggcttggttcacgcctgtaatcccagcactttggggagctgaggtgggcagatcaattgaggccaggagtttgagaccagccacaccaacatggtgaaaccttgtctctactaaaaatgcaaaaattaggcatggtggtacatgcctgtagtcccagctactcgggaggctgaggcaggagaatcgcttgaacctgagaggtggaggttgcagtgagtcgagatcgcaccgttgcactccagcctgggcgacaaagcaagactctgctcaaaaaaacaaaaaaaaaaacaaaaaaagaaaaggaaagtaaaacaataaatgatggtggtcctgtgatttgctgttggtctacgtgaggccctgtgcatgggatttcacaaacatgttcttgaatcctctcaaaaccagcttgaaggttggtggtgtctccctggtgtgacagggtctgtcatacagctggcattcagcaacaacaacaacaaaaatagaaatgggagtctcgctatgttgcccaggctggtctccaactcctgggctcaagtgaccctcctgtctcagcatcctgagtagctggaatacaggtacacacttccacacccaggctatcaactgttttttaaatgaataaatcaaattagtcaattttacagaaggggaaagtgaggcttggagagagactttgatggacataggacttgcggagttttatagattcttagtttttgttcgtttgtttgtttttgtttttgagacagagtcttgctctgttgcctaggctggagtgcagtggcgtgatcttgtcttactgcaacctctgcctcccaggcttaagccgttctcctgcctcagcctcccaagtagctgggactatagatgcgtgccaccacacctggctaatttttgcatttttagtagagacagggttaaatgttaggcagactggtcttaaactcctgacctcaggtgatctggctgcctcggccttccaaagtgctgggattacaggtgtgagccactgtgcccggccttttttttttgtttttctttgagatgaaaagtcactcttgtcgcccaggctggagtgcaatggtacgatctcagctcatggcaacctccgcttccagaattcaagcaattctcccgcctcagcctcccaagtagctgggattacaggcgcccgccaccatgcgcagataatttattttattttattttatttattattattattattattattattattatttttgagatggagtttcgctctgtcgcccaggctggagggcagtgacgcgatctcacctcactgcaagctccgcctcccgggttcacaccattctcctgcctcagcctcccgagtagctgggactacaggcacctgccaccacacccggctaactttttgtatttttagtagagatggggtttcaccatgttagccaggatggtctcgatctcctgacctcgtgatccgcccgcctcggcctcccaaaagtgttgggattacaggtgtgagccaccgcgtccggccaatttttttatttttagtagagacgaggtttcaccatgttgcccaggctggttgctaactcctgacctcaggtgatcagcccgcctcggcctcccaaaatgctgggattacaggcgtgagcccctgcacctggccagatttagttttgggtgggccaagatcttgtgcctctgatacagtcattttccatatcatatttttgtttctggggttctgctgagggcagcgtgatttcatcacttgaacactttgcggaactgggcaggaagcactctgcccatttcatagatgggcaaactgagcctccgtcctgtgcctcttcgggttggggtggataagagcaaaacagggcagggagtggggaagctctgggaggccttgatcagagcgctctggctctgccactttccagcttggtggtctcctgcgtcctcacgtgggcagggggattgagacctgcagctgggttggcatgaggtggatgaagctgctgggcaagtgtgggattgattttctgtggggactcgagtggaatgtttctctgttggccca

SEQ ID NO: 2 - DNA, 9821 bp, Homo sapiens
gggagatgcaggcggggagacacaaggtagaaggggcaaagtcctcacctaggaccttgagggagttaatgtgtaatattctaggatataagcttgaccacgagttgagaccctgagcacaggcctccaggagccgctgggagctgccgccaggagctgtcaccatgacggtgaggacactagccccctgctgcctgccccactctgttcatctttgtctttgcctgggtgggggcttttagggaaaaccattgctgtccctctctgggcctcagtttccccatctgtgcagcaaagaagttggacagaggtctttttttaaaaaacagcatcttgggccaggcgtggtggctcctgcttgtaatcccagcactttgggaggccgaggctggtggatcatctgaggttgggagtttgagaccagcctgaccaacatggagaaaccccgtctctactaaaaaaatacaaaattggctaggcctggtggcacatgcctgtaatcccagctaatggggaggctgaggcaggagaatcacttggacctgggaggcagaggttatggtgagccgagattgtgccattgcactccagcctgggcaacaagagtgagactccatctcaaaacaacaacaacaatacagcatcttgctctgtcaccaggtggagtgcagtggtggcaatcataactcactacggacttgacctccttggcttaaatgatcctcccacctcagcctcttgagtagctgggaccccaggcactcactaccacactggctaattttgtttgtttcttttctttctctttttttttttttttttgagatggagtctcgctctgttgcccaggctggagtgcagtggcccgatctcagctcactgcaacctctgctgcctgggttcaagcaattctctggtctcagcctcccaagtagctgggattacaggtatgtgtcaccacacctggctaatttttttttttttgttgagatggagtttctgttgcccaggctggagtgcaatggcacgatctcggctcaccacaacttccacctcccaggttcaagcgattctcctgcctcagcctcctgagtagcagggattacaggcatgggccaccacacccgattaattttgtatttttagtagagatggggtttctccatgttggtcaggctggtcttgaactcctgatctcaggtgatccacctgccttggcctcccaaagtgctgggattacaggtgtgagccactgctcctggcctaatttttgtatttttaaagtagagacagggtttcaccatgttggtcaggctgatctcgaactcctgacctcaggtgatccgcccaccttggcctcccaaagtgctgggattacaggtgtgagccaccccacccagcttatttcttatttttcgtagagatgaggtctcactatgttgctcaggctgatatcaaactcctgggttcaagggatcctcctgccttggcctctcgaagtgctgagattacaggtgtgagccactgtgcctggcctccattgatctttatagagataaaaaaaaatctcagcttgggcaatatagtgagaccttttctgctacaggtgcatgccactacgctttgccttaaaaaattagtgggggtagcggcacactcctcagccttgggaggctgaggatcacttgagcccaggaggtcgaggctacagtgagccgtaattgcactactgtactccagcctgggcaacagagtgagaccttgtctcatatacccacacacaaaacccaagtcttggagagcaaattgcccaaggccacaagctgcaaatcacaaggggttgagtggattcccactgaggtctctgattcgttgattctacaccagactctgccacagctttactgtgtggccttggccaagtcactgaccgtctctgagccccagtcttccttacatctgtggaaggggatcacaggctgcctcttctgaggattagatggtgtattcattgcctagggctgcaataacaaattaccaccaaattgtgggtggcttcacacgatagacgtttgttctgtcttggttttggtgactagaaacctgaaaccaaggtgctacagggctacgctcctgctgaaggcgcaaggggagggttctttcttgcctcttccagcttctggtggctcctcgcattccttggcttgcatcactccaatctctgcctccaacttcacgtggactcctctgtgtgtctccgtctctgtgtctatatttctctcctcttatgagaacactggtcgtattggatttaggaccaaccctaaaccagtatgacctcttaactcgattacatatgcaaaggaactatttttaaataggtcacattgaggctgggcggggtggctcacccctgtaatcccagcactttgggaggccgaggcaggcggatcacttgagatcaggagttcaagaccagcctggccaacatggtgaaaccctgtctgtactgaaaatacaaaaacaaaaaagaagaagaagaagaaaaaaaaattagacagatgttgtggtgggcacccgtaatcccagctacttgggaggctgaggcaggagaatcggttgaacccgggaggcagaggttgcagtgagccgagatcgagccactgtactccagcctaggtgacagagtgagacttcatctcaaaaaaaaaaaaaaaggtcacattgacaggttccaggtggacatgaattttcgggggacgctattcaagtgcaggggggatgcaggatgtgaatgtgccaggggtcctgcgtggaagggtctatgccctcatcaccctctgcctctcggggaggactgctgtggcccacggactctccccaccttctctttcctggtcatctcacctctgccttttctttcctctctctccagctccagaggccatatcatccaaatcccttatacgacagataagggaaccaaggcccagaaaggggctaagctggccccaggcccctctgccaattaggggcagagtcggcactagagtctgggcccccaactccccacccccccagctctagggacgaccacacccccacccagttctgcctgtctctctctgcgcctttgactctgttgggtggggacaaggctcccgggcctgcaccctcccgcagctctcagcatccctatttgtccaagtgcacccctgaccctggacttccgagtgcttctgccctgcagcagcccccacctctatccttggggtttgagctttgctgtttcagtcaggcagcccccaggagctgcaaggggagtgtgggtgcttctcttagtccaggcccagctcccctatcctggcctgactgttgcagggctcggggtgtgggcacaggctgctggcaggaggcagggagccatctcctgatgcttggtgttagacgtgtgtgtgcgcagggcacacgtctgtgagtgtctgtgtggcgggcacacctgtcttctgtttcttgtttgagccccttttggactgtcctcactggataacctcatctcccagagataatggtctttgtcagtgagagactgatttttttttttttttttttttttttgagacggagtctcgctctgtcgcccaggctggagtgcagtggcgccatcttggctcactgcaagcaccgcctcccgggttcacgccattctcctgcctcagcctcccgagtagctgggactacaggcgcctgccaccacgcccggctaattttttgtatttttagtagagacagggtttcaccgtgttagccaggatgatctcactctcctgacctcgtgatccgcccccctcggcctcccaaagtgctgggattacaggtgtgagccaccgcccctggccagcaagagactgattttaatcccgtctgtctggctccaaaatctggacccaaccccgttgtgttaagcaaagacatggggagttaggtgtccagcctccaaaccccactttctctaaagcagggaggttttgctcccaggagacaacggaccctgtctggagacattcttggttgtcaccgctcaggggagggtgtcactgacatccagtgggtagaggccaggaatactgctcaacatcctacaacacaagagacagaccccaacaaagaaatgcctgccccaaacgtccagacggccaaggctgagaagctctggtctgagcagcctcctgtctgacatgccgccgtcatggcccgctgtcctgggttaagcattgctgcctcctccaggcgtctcttataaaatgtactgccaggccgggcacagtggcttacacctgtaatcccaacactttgggaggccgaggtgggaggatcctttgagctcaggaggtcgaggctggcctggacaacatagtgagaccccatctgaaaaaaaaaaaaatcagctgggccaggtgggatgcctgtggtccaggctacctggggggctgaggtgggaggattgcttgagcccaagaggtcaaggctgtagtgagctctgatcatatcactgcactctagcctgggtgacagagcaagacctttaaaaaaaaatgtattaccggctgaggcaggaggaccacttgagggtcaggagttccagagcagcctgggtaacatagcaaaaccctatctctacaaaaattttaaaaattagctgggcatggtggcacacgtctatagtcgtagctacttaggaggctgaggcaggagaatcgtttgagctcaggaggctatgaggctgcatgcagtgagttataatcgtgctactgcactccatcttgggtgacagagcaagaccctgtctcaaaaaaaaaaaaaaaagaaagaaaagaaaaaaaatgctgggtgtggtatctcacccctataatcccagaactttgggaggcccagaggggaggatcacttgaggtcaggagttcgagaccaacctggccaaagtggtgaaaccccgtctctactaaaaatacaaagaaaattagctagatatggtggtgggagcctgtaatctcagtgactcgggaggctgaggcaggataattgcttgaatctgggaagtggaggttgcagtgagccaagattgcaccactgcactccagcatgggtgacagagtgagactccatctcaacatctcaaaaaaaaaaaaaaaagaacttactgcctgtggaagagttgagcaatacctaacaacctacccctacatgtgaccaaccagcgggtcacttcctcctctgcagagaggaggcggctgccagcgagagggcactgagggtcctcccatggccactgcccccttgacttctggcaaagtgccccagtccaatgagctcattcagggcatctcagatcatgctttttctggaaataaaaagtcagtgagcagaactcccacaatgtaaaagtgtcctcccataagttgttctaaatctttggtgcctgttgcgtcctggtcagaccaaccctcaccctctggtcatagatgcgaaaactggtcttgggtaatgagtttttttttttttttttttttagacagagtcttgctctgtcgcctggcacaatctcggctcactgcaacctccacctcctgggttcaagcgattctcttgcctcagcctcccaagtagctgggacgacaggcatgtgccaccacacccggctaatttttgtatttttaatagagacagggtttctccatgttggccaggccagtctcaaactcctgacctcaggtgatccacccgactcagcctcccaaagtgctgggattacaggcgtgagccaccgctcccagccctgggtaatgagctttgaaaacccagcttagaaatcttccctagtaaccatcgtgaggctagaggaggctcctactgtacagaaattcaggtgctgctttcctatggaaaataaggagcagatgaatcttaacaacaagtaatcaaaatgatggtcatttgggcagaccactgtccagaaaaaaagaaaaaatttaaaaaagaaaattaaggctgggcttggttcacgcctgtaatcccagcactttggggagctgaggtgggcagatcaattgaggccaggagtttgagaccagccacaccaacatggtgaaaccttgtctctactaaaaatgcaaaaattaggcatggtggtacatgcctgtagtcccagctactcgggaggctgaggcaggagaatcgcttgaacctgagaggtggaggttgcagtgagtcgagatcgcaccgttgcactccagcctgggcgacaaagcaagactctgctcaaaaaaacaaaaaaaaaaacaaaaaaagaaaaggaaagtaaaacaataaatgatggtggtcctgtgatttgctgttggtctacgtgaggccctgtgcatgggatttcacaaacatgttcttgaatcctctcaaaaccagcttgaaggttggtggtgtctccctggtgtgacagggtctgtcatacagctggcattcagcaacaacaacaacaaaaatagaaatgggagtctcgctatgttgcccaggctggtctccaactcctgggctcaagtgaccctcctgtctcagcatcctgagtagctggaatacaggtacacacttccacacccaggctatcaactgttttttaaatgaataaatcaaattagtcaattttacagaaggggaaagtgaggcttggagagagactttgatggacataggacttgcggagttttatagattcttagtttttgttcgtttgtttgtttttgtttttgagacagagtcttgctctgttgcctaggctggagtgcagtggcgtgatcttgtcttactgcaacctctgcctcccaggcttaagccgttctcctgcctcagcctcccaagtagctgggactatagatgcgtgccaccacacctggctaatttttgcatttttagtagagacagggttaaatgttaggcagactggtcttaaactcctgacctcaggtgatctggctgcctcggccttccaaagtgctgggattacaggtgtgagccactgtgcccggccttttttttttgtttttctttgagatgaaaagtcactcttgtcgcccaggctggagtgcaatggtacgatctcagctcatggcaacctccgcttccagaattcaagcaattctcccgcctcagcctcccaagtagctgggattacaggcgcccgccaccatgcgcagataatttattttattttattttatttattattattattattattattattattatttttgagatggagtttcgctctgtcgcccaggctggagggcagtgacgcgatctcacctcactgcaagctccgcctcccgggttcacaccattctcctgcctcagcctcccgagtagctgggactacaggcacctgccaccacacccggctaactttttgtatttttagtagagatggggtttcaccatgttagccaggatggtctcgatctcctgacctcgtgatccgcccgcctcggcctcccaaaagtgttgggattacaggtgtgagccaccgcgtccggccaatttttttatttttagtagagacgaggtttcaccatgttgcccaggctggttgctaactcctgacctcaggtgatcagcccgcctcggcctcccaaaatgctgggattacaggcgtgagcccctgcacctggccagatttagttttgggtgggccaagatcttgtgcctctgatacagtcattttccatatcatatttttgtttctggggttctgctgagggcagcgtgatttcatcacttgaacactttgcggaactgggcaggaagcactctgcccatttcatagatgggcaaactgagcctccgtcctgtgcctcttcgggttggggtggataagagcaaaacagggcagggagtggggaagctctgggaggccttgatcagagcgctctggctctgccactttccagcttggtggtctcctgcgtcctcacgtgggcagggggattgagacctgcagctgggttggcatgaggtggatgaagctgctgggcaagtgtgggattgattttctgtggggactcgagtggaatgtttctctgttggcccagggggaacttgaggttaagaacatggacatgaagccggggtcaaccctgaagatcacaggcagcatcgccgatggcactgatgggtgagcaaggtttcagggttgggggagtctgcaggcccggaataggcagggcgggtggggcaggcagggcagccctgtgaagtgctcaggcaagagggacgtcaggccaatgggccctttttcacacccttctccccacacccctgctggcccccacttcatgtctgaggctaggtttggggacctgcagaatttcagagttgatgccatatgctctattcttttgccccaacagccattgaaggggcaggtggagaagcccctggaactctgtctggccccctgcggggcaggtgcctctagggaacgcccaaatccccagagacaccaccctctttacccagcagaatggccacaggctggcatttcatgagcattaaaccagggcagccaccaggggaggctgagtggtctcgctggcatcctcttggttagaaccagcggcctcaccacctccgtgagtcacagtccagcgaaaggctctctcgcctgcagaacatgtcagcgcatcttggaactgtgctttatctacttttggttagagagggggcgggcaggtgcatgccataggagctaagggaaaagtgacttatttctcctacttgggtccctcaagtttgtcaaaatgtgtgatacccttggtctgagactcccaaatgaagacaccccatgacccagaatgccccactttcaggaaccctgcaggtctagcccaggctcctgtagtgatcttgccaagaagtcatacaaccccggttgcacacccatagtgacagggagctcaccaccttaggttggctgctggtggctaaatttaataggtcttcagatatctaagagatagcatttctctctcccaggagagccacccccaattcccgaagctgtcactatcagttacccttctctcaacagcgtgatccctgctccaaatggaatgtgctaccacagtgctaagtctgagcaggttgttacctcccttgttttaaggcacagatctcaactaacacaagctttgattcttccagcttgtggtcaaccaaggtcctccaacccaagctgctttatccaggcctgagccctgaacctcacctgctaccccttctcctgcagctttgtaattaatctgggccaggggacagacaagctgaacctgcatttcaaccctcgcttcagcgaatccaccattgtctgcaactcattggacggcagcaactgggggcaagaacaacgggaagatcacctgtgcttcagcccagggtcagaggtcaaggtgaggtcaaagggggaaagggcactggggtgatgtcaaggggagggcccagatggaagagagcctggcctggacacaggtgctggccttgtttgagccatcaggcactgccctggcccatttccagggcctcctgcctccttgacaccctccctccccacagttcacagtgacctttgagagtgacaaattcaaggtgaagctgccagatgggcacgagctgacttttcccaacaggctgggtcacagccacctgagctacctgagcgtaaggggcgggttcaacatgtcctctttcaagttaaaagaataaaagacttccagccg

SEQ ID NO: 3 - DNA, 558 bp, Homo sapiens
cccccccagctctagggacgaccacacccccacccagttctgcctgtctctctctgcgcctttgactctgttgggtggggacaaggctcccgggcctgcaccctcccgcagctctcagcatccctatttgtccaagtgcacccctgaccctggacttccgagtgcttctgccctgcagcagcccccacctctatccttggggtttgagctttgctgtttcagtcaggcagcccccaggagctgcaaggggagtgtgggtgcttctcttagtccaggcccagctcccctatcctggcctgactgttgcagggctcggggtgtgggcacaggctgctggcaggaggcagggagccatctcctgatgcttggtgttagaygtgtgtgtgcgcagggcacacgtctgtgagtgtctgtgtggcgggcacacctgtcttctgtttcttgtttgagccccttttggactgtcctcactggataacctcatctcccagagataatggtctttgtcagtgagagactgatttttttttttttttttttttttttgagacggagtct

SEQ ID NO: 4 - DNA, 19 bp, Homo sapiens
ctgcgcctttgactctgtt

SEQ ID NO: 5 - DNA, 20 bp, Homo sapiens
tctttgtcagtgagagactg

SEQ ID NO: 6 - DNA, 20 bp, Homo sapiens
cctatcctggcctgactgtt